(19) United States
(12) Patent Application Publication
(10) Pub. No.: US 2009/0090365 A1
(43) Pub. Date: Apr. 9, 2009

(54) BALLOON CUFF TRACHEOSTOMY TUBE WITH GREATER EASE OF INSERTION

(76) Inventors: Brian J. Cuevas, Cumming, GA (US); James F. Schumacher, Cumming, GA (US); Michael A. Kenowski, Alpharetta, GA (US); Sam C. Chan, Alpharetta, GA (US); Ryan C. Frank, Westerville, OH (US)

Correspondence Address: KIMBERLY-CLARK WORLDWIDE, INC.;Catherine E. Wolf, 401 NORTH LAKE STREET, NEENAH, WI, 54956, US

(21) Appl. No.: 12/206,560

(22) Filed: Sep. 8, 2008

Related U.S. Application Data
(60) Provisional application No. 60/994,664, filed on Sep. 20, 2007.

(51) Int. Cl. A62B 9/06; A61M 16/00

(52) U.S. Cl. 128/207.14; 128/200.26

(57) ABSTRACT

There is provided a balloon cuffed tracheostomy tube with a balloon designed so as to enhance the tube's anchorability without sealing the tracheal stoma and to allow for an easier insertion into the trachea than a comparable tube with a thicker balloon. The tracheostomy tube device includes a conventional hollow tube having a proximal end portion, a distal end portion, and a bend region intermediate of the end portions. The distal end portion of the tube is arranged for insertion through a patient's throat and tracheal stoma and into the tracheal lumen. The device further includes an inflatable balloon enveloping a portion of the tube. More particularly, the balloon is equal to or less than 30 microns in thickness, allowing for a greater ease of insertion and insertion through a smaller stoma opening, than a comparable tube with a balloon having a thickness greater than 30 microns.

This application claims the benefit of commonly assigned U.S. provisional application 60/994,664, filed Sep. 20, 2007 and having attorney docket number 64391725U.S.01.

### BACKGROUND OF THE INVENTION

A tracheostomy procedure involves making a small horizontal incision in the skin of the neck to grant access to the trachea. The trachea proper is not cut since, because of the uniquely flexible and elastic nature of the trachea, it has been found that healing is much faster if only a small puncture hole is made in the tracheal wall and the hole or stoma dilated, rather than cutting the tracheal wall. After the initial puncturing of the trachea, it is dilated to increase the size of the opening to a size sufficient to allow for the introduction of a tracheostomy tube. This dilation may be performed using a series of dilators, each larger than the one before, as the procedure was performed for many years. The dilation may alternatively be performed using a single dilator such as the Cook Medical Inc. Blue Rhino® dilator (see also U.S. Pat. No. 6,637,435). Dilation of the tracheal stoma may also be performed through the use of balloon dilators that are inserted into the stoma and that then expand as they are pressurized with an externally supplied fluid such as air. After the stoma is sufficiently dilated, a tracheostomy (trach) tube device may be inserted into the trachea through the stoma and placed in service using a ventilator.

Ventilators or respirators are used for mechanical ventilation of the lungs of a patient in a medical setting. The ventilator unit is connected to a hose set; the ventilation tubing or tubing circuit, delivering the ventilation gas to the patient. At the patient end, the ventilation tubing is connected to the trach tube that has been placed in the trachea through the stoma described above.

The trach tube device typically has a tube or catheter, granting direct and secure access to the lower airways of a patient and an inflated sealing balloon element, or “cuff”. The balloon creates a seal between the tracheal wall and tracheal ventilation tube shaft, permitting positive pressure ventilation of the lungs. The balloon adheres to the internal lining of the trachea in its generally cross-sectional dimension in order to prevent air insufflated by the respirator into the patient from escaping to the environment through the tracheostomy or the larynx and pharynx. This enables the air to reach the lower airways and eventually the pulmonary alveoli. The balloon also aids in supporting the tube inside the trachea. Conventional tracheostomy device designs, however, may contribute to a variety of frequent complications associated with tracheostomies.

Conventional trach tubes are not well anchored within the body and as a result, the tube can move a great deal inside the airway, as well as through the tracheal stoma and the wound. This instability and lack of anchorage can lead to a number of problems such as irritation of the tracheal tissue by the distal end of the trach tube. Designs of tracheostomy tubes devices with balloons that expand to anchor the tube have been proposed. An example of such a balloon is shown in U.S. Pat. No. 6,612,305 “Integral Balloon Tracheostomy Tube” and illustrated in FIG. 9. While these tracheostomy tube devices may provide suitable anchoring, the balloons appear to seal the tracheal stoma thus limiting access to that region.

Conventional tracheostomy balloons may create further complications because they can be quite difficult to insert through the tracheal stoma. These balloons tend to bind or catch on the stoma and/or the tracheal rings as the medical professional inserts the device into the trachea because the balloons are typically quite thick. These thick balloons create, even in the un-inflated state, a cross section or profile much greater than that of the tube alone. The trauma to the patient caused by inserting these large balloons should be avoided if possible as it can negatively affect patient comfort and may contribute to inflammation and other adverse results. One way of avoiding this problem is by simply making the opening or stoma in the trachea larger prior to insertion of the tracheostomy device. While this approach would certainly be successful in aiding insertion, it should be clear that this approach would cause greater trauma than would the making of a smaller opening.

It would therefore be desirable to provide a tracheostomy tube and balloon design that is more stable within the patient than currently available tubes and which may be inserted with less force or trauma or through a smaller stoma.

### SUMMARY

The subject of the present disclosure relates to a balloon cuffed tracheostomy tube with a balloon designed so as to enhance the tube's anchorability without sealing the tracheal stoma and which is easier to insert into the stoma than conventional tracheostomy devices and/or which may be inserted through a smaller stoma.

The device further includes means for inflating and deflating the balloon.

These means for inflating and deflating the balloon may be conventional flexible conduits and fittings.

The tracheostomy tube device includes a hollow tube or catheter having a proximal end portion, a distal end portion, and a bend region intermediate of the end portions. The distal end portion of the tube is arranged for insertion through a stoma in a patients' throat and into the tracheal lumen such that the distal end portion of the tube extends in a first direction within the tracheal lumen when the proximal end portion extends in a second direction through the tracheal stoma. The proximal end portion defines a proximal plane of the device.

The device further includes an inflatable balloon enveloping a portion of the tube. More particularly, the balloon has a distal balloon portion substantially centered about and attached to the distal end portion of the tube. The balloon also has a proximal balloon portion attached to the bend region of the tube and positioned substantially off-center about the bend region below the proximal plane of the device. Upon inflation, this configuration provides for expansion of the balloon around the distal end portion of the tube and the proximal end portion of the tube below the proximal plane of the device to seal the trachea below the tracheal stoma and avoid sealing the trachea above the tracheal stoma. Desirably, this configuration of the balloon will allow secretions to exit the stoma.

The balloon has a wall thickness that is desirably 30 microns or less. Such a thickness allows the balloon to lie tight to the shaft or tube and enter the stoma with less force or trauma than a comparable tube with a thicker balloon. This also allows the balloon of this disclosure to enter a stoma that is smaller than the stoma required for a thicker balloon on a comparable tube. The balloon component may be formed from thermoplastic polyurethane polymers, thermoplastic polyolefin elastomers, thermoplastic polyolefin block copolymers, SBS di-block elastomers, SEBS tri-block elastomers and blends and mixtures thereof.

In another aspect of the invention, the tracheostomy tube device may further include a flexible conduit along the tube and the balloon such that secretions eventually accumulating cranially to the tracheal stoma can be removed. These secretions, if not removed, provide a medium in which bacteria may grow.

Furthermore, should these secretions pass by the balloon and proceed into the lungs, they may result in the patent contracting pneumonia. This condition, known as ventilator acquired pneumonia or VAP is a significant and growing problem in many hospitals.

According to one aspect of the disclosure the inflatable balloon may include a distal end, a distal attachment zone, a proximal end, a proximal attachment zone, an upper region and a lower region, wherein the upper region has a thickness of from about 15 to about 30 micrometers and the lower region has a thickness of from about 5 to about 15 micrometers.

### BRIEF DESCRIPTION OF THE DRAWINGS

FIG. 1 is a drawing of a deflated conventional balloon cuff having a conventional wall thickness.

FIG. 2 is a drawing of an exemplary deflated thin balloon cuff according to an aspect of the present disclosure.

FIG. 3 is an illustration of an exemplary device in which the balloon is inflated to seal the trachea in the region below the tracheal stoma while avoiding sealing the trachea in the region above the tracheal stoma.

FIG. 4 is an illustration of an exemplary balloon cuffed tracheostomy tube designed so as to enhance the tube's anchorability without completely sealing the tracheal stoma.

FIG. 5 is a perspective view of an exemplary inflatable balloon component.

FIG. 6 which is a side view of the exemplary inflatable balloon component of FIG. 5.

FIG. 7 is a perspective view of another embodiment of an exemplary inflatable balloon component.

FIG. 8 which is a side view of the embodiment of an exemplary inflatable balloon component of FIG. 7.

FIG. 9 is a drawing of a tracheostomy tube as described in U.S. Pat. No. 6,612,305.

### DETAILED DESCRIPTION

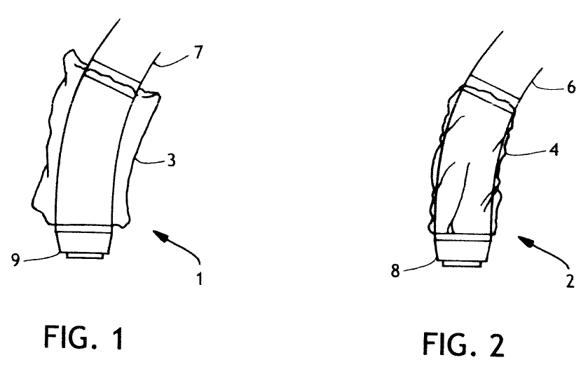

FIG. 1 is an illustration of the lower end of a conventional cuffed tracheostomy tube device 1 having a balloon 3 disposed on a tube 7 the distal end 9 of which is adapted to be inserted into a tracheal stoma. The balloon, prior to insertion into the stoma, is un-inflated and the membrane out of which the balloon is composed hangs loosely about the tube, as shown.

FIG. 2 is a drawing of the lower end of a tracheostomy tube device 2 according to the instant disclosure. The device 2 has a balloon 4 disposed on a tube 6, the distal end 8 of which is adapted to be inserted into a tracheal stoma. As in the conventional device, the device according to this disclosure, prior to insertion into the stoma, is un-inflated. The membrane of which the balloon is made hangs loosely about the tube. In contrast to FIG. 1, however, the balloon as shown in FIG. 2, by virtue of its thinness, lies tight to the shaft of the tube. This attribute of the disclosed device provides an advantage in ease of insertion of the device into a stoma and/or allows the disclosed device to enter a smaller stoma than a comparable device with a thicker balloon. Desirably, the balloon of the present invention has a balloon wall thickness that is less than 30 microns. More desirably, the balloon has a balloon wall thickness of less than about 25 microns. Even more desirably, the balloon has a balloon wall thickness of less than about 20 microns. It is contemplated that the balloon wall thickness may be less than 15 microns or even from about 5 microns to about 10 microns in thickness.

The measurement of balloon wall thicknesses may be made using a Litematic device. An exemplary device is the series 318 Model VL-50A by Mitutoyo America Corporation. According to the manufacturer, the Litematic device measures thicknesses between 0 and 50.8 mm with a resolution of 0.01 micron, using a probe tip and an inflexible ceramic base. The measuring force used is 0.01N (1 gram). The probe tip used for testing herein was a 3 mm diameter carbide ball contact point which was provided as the “standard” probe tip with the Litematic device.

Strips of single-ply foils or membranes may be used to determine the thickness of each sample. Balloon specimens (not attached to a trach tube) from each sample may be cut to prepare the strips: first the ends should be cut off to leave a uniform band of about 30 mm in width; then each band should be cut in the width direction to form a strip. Thickness measurements at 10 locations along the length of each strip should be made, the individual measurements of strips for each sample (with at least 6 strips measured) should be averaged together, and the respective standard deviations calculated.

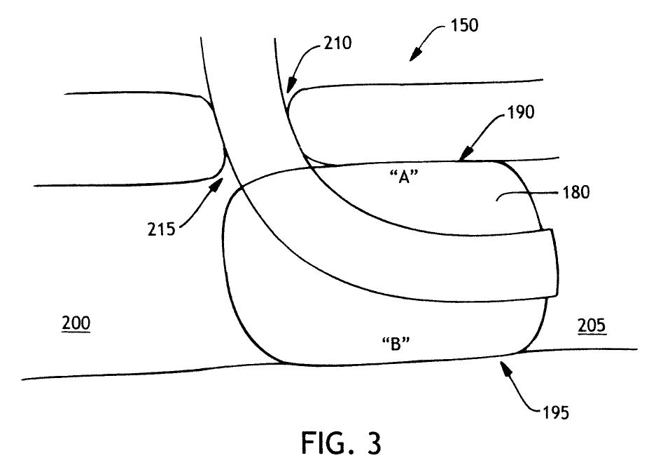

The balloon disclosed in the Summary above performs its normal function of obdurating the trachea in such a manner as to allow secretions to reach the stoma. As discussed above, balloons according to, for example, U.S. Pat. No. 6,612,305, provide anchoring of the balloon in the trachea but appears to block access to the tracheal stoma for secretions to exit. These secretions may harbor bacteria or promote ventilator acquired pneumonia. FIG. 3 is an illustration of an exemplary device in which the balloon is inflated to seal the trachea in the region below the tracheal stoma while avoiding completely sealing the tracheal stoma.

As can be seen in FIG. 3, it is desired that the first or anterior portion “A” of the balloon 180 is the portion of the balloon contacting the upper portion of a cross-sectional region of the tracheal lumen 200 and the second or posterior portion “B” of the balloon is the portion of the balloon contacting the lower portion of the same cross-sectional region of the tracheal lumen. The inflated balloon 180 is adapted to obdurate or seal the trachea (i.e., the tracheal lumen 200) in the region 250 below the tracheal stoma 210 and avoid sealing the trachea in the region above the tracheal stoma. Desirably, this configuration of the balloon allows secretions to exit the stoma at opening 215.

It is further desirable that the balloon have a membrane of varying thickness in different regions of the balloon. For example, the upper portion “A” of the balloon 180 desirably has a thickness of from about 15 to about 30 micrometers and the lower portion “B” desirably has a thickness of from about 5 to about 15 micrometers. Although the inventors should not be held to a particular theory of operation, it is generally thought that having the relatively thinner second portion “B” of the balloon contacting the lower wall 195 of the trachea will provide a better seal in that region where secretions may be more prone to collect due to gravity when a patient is resting horizontally on his back. The relatively thicker first portion “A” of the balloon is in contact with the upper wall 190 of the trachea where secretions may be less prone to collect due to gravity when a patient is resting horizontally on his back.

A balloon having a membrane of varying thickness may be produced, for example, by expanding a raw plastic tube having an asymmetric wall thickness and/or by making the balloon in a mold that is asymmetric. When such a tube is preheated in a mold to a temperature sufficient to soften the material of the tube and inflated with a gas to generally uniformly stretch the material of the tube, the tube forms a balloon also having asymmetric wall thickness. Likewise, when the tube is placed in a mold so that the tube does not pass through the centerline of the balloon to be blown, the resulting balloon has walls that vary in thickness. Desirably, the upper region has a thickness of from about 15 to about 30 micrometers and the lower region has a thickness of from about 5 to about 15 micrometers. The resulting balloon may subsequently be placed on a tube by means known to those skilled in the art.

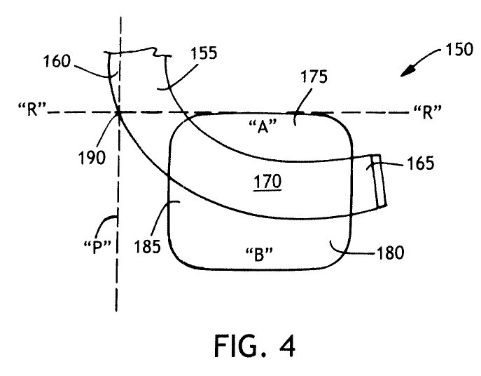

In another informative view similar to FIG. 3, FIG. 4 shows a tracheostomy device in relation to the upper portion of the trachea and the upper edge of the stoma. The tracheostomy tube device 150 includes a hollow tube 155 having a proximal end portion 160, a distal end portion 165, and a bend region 170 intermediate of the end portions. The distal end portion of the tube is arranged for insertion through a tracheal stoma and into the tracheal lumen such that the distal end portion 165 of the tube extends in a first direction within the tracheal lumen when the proximal end portion 160 extends in a second direction through the tracheal stoma, outside the body and ultimately to the ventilator.

FIG. 4 illustrates that the proximal end portion 160 defines a proximal plane of the device “P”. Generally speaking, the proximal plane of the device is a plane that runs along the portion of the tube as it passes the stoma just inside the tracheal lumen that is closest to the patient's head, i.e. the edge of the stoma in the cranial direction. The disclosed device further includes an inflatable balloon 175 enveloping a portion of the tube 155. As illustrated, the balloon 175 has a distal balloon portion 180 substantially centered about and attached to the distal end portion of the tube 165. The balloon also has a proximal balloon portion 185 attached to the bend region of the tube and positioned substantially off-center about the bend region 170 below the proximal plane of the device “P”.

After insertion of the devise illustrated in FIG. 4, the balloon is inflated. Upon inflation, this configuration provides for expansion of the balloon 175 around the distal end portion of the tube 165 and the proximal end portion of the tube 160 below the proximal plane of the device “P” to seal the trachea below the tracheal stoma and avoid sealing the trachea above the tracheal stoma. Secretions should have easy access to the stoma where they may be removed by absorption into a stoma pad placed below the flange attached to conventional trach tubes at the throat.

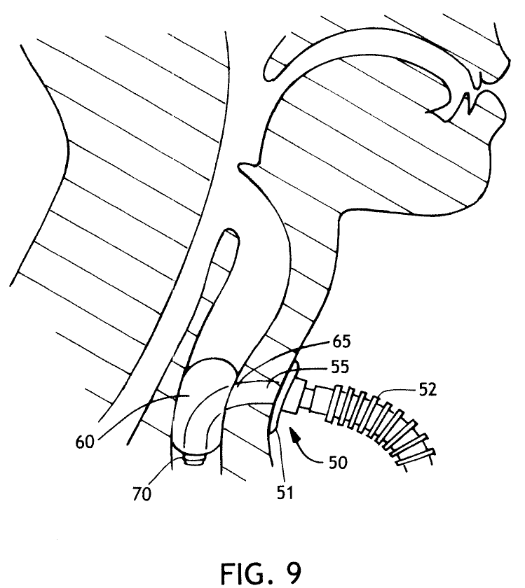

More particularly, a stoma pad or other secretion media may be placed between the skin of the throat and the trach tube flange (see FIG. 9). This pad may be removed and disposed of on a regular basis and replaced with a fresh pad so that bacteria does not accumulate above the stoma.

With further reference to FIG. 4 it should be noted that while various curvatures of the bend region of conventional hollow tracheal tubes may result in the proximal plane being located slightly more or slightly less forward, the proximal plane of the device “P” may be readily determined from a reference line “R” running parallel to the upper surface of the inflatable balloon 175 (while inflated). The proximal plane of the device “P” is the plane that is perpendicular to the reference line “R” and which passes through the point 190 where the reference line “R” intersects with the most proximal or outermost portion of the tube 155. This is generally thought to correspond to a plane that runs along the portion of the tube as it passes the stoma just inside the tracheal lumen that is closest to the patient's head, i.e. the edge of the stoma in the cranial direction.

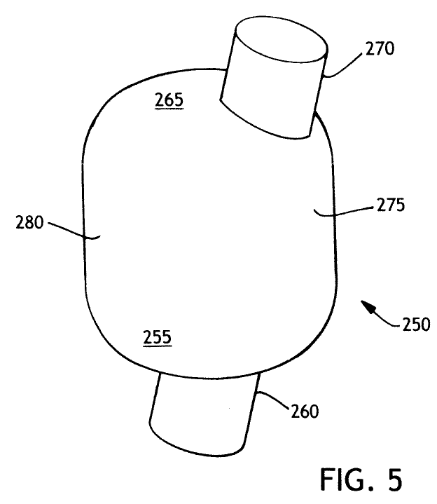

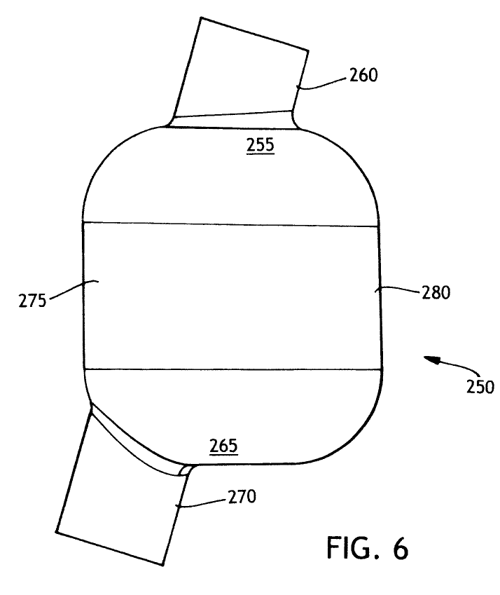

Keeping in mind the reference points discussed in relation to FIG. 4, FIG. 5 is a perspective view of the resulting inflatable balloon component 250. FIG. 6 is a side view of the same balloon. This inflatable balloon component may include a distal end 255, a distal attachment zone 260, a proximal end 265, a proximal attachment zone 270, an upper region 275 and a lower region 280. According to an embodiment of the disclosure and as noted above, the upper region desirably has a thickness of from about 15 to about 30 micrometers and the lower region desirably has a thickness of from about 5 to about 15 micrometers. Extrapolating the locations of P, R and the proximal tube results in a location for their intersection at a point relatively far from the proximal end 265 of the balloon 250. Nevertheless, this configuration allows for an opening between the proximal end of the balloon and the cranial edge of the stoma.

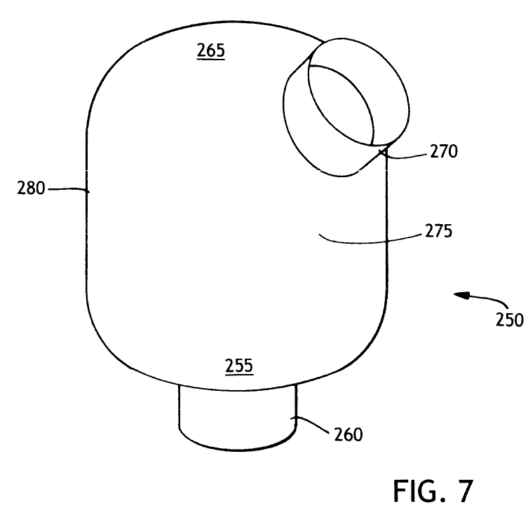

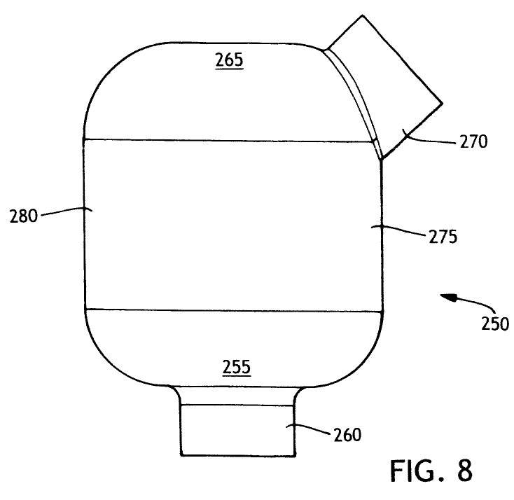

FIG. 7 is a perspective view of another embodiment of the resulting inflatable balloon component 250 and FIG. 8 is a side view of the same balloon. As can be seen in FIGS. 7 and 8, the inflatable balloon component may include a distal end 255, a distal attachment zone 260, a proximal end 265, a proximal attachment zone 270, an upper region 275 and a lower region 280. According to an embodiment of the instant disclosure, the upper region desirably has a thickness of from about 15 to about 30 micrometers and the lower region desirably has a thickness of from about 5 to about 15 micrometers. Extrapolating the locations of P, R and the proximal tube results in a location for their intersection at a point relatively near to the proximal end 265 of the balloon 250. This configuration also clearly allows for a relatively large opening between the proximal end of the balloon and the cranial edge of the stoma.

In reference to FIGS. 5 through 8 generally, the dimensions from the upper region 275 to the lower region 280 may range from about 50 millimeters to about 25 millimeters and may desirably be between about 35 millimeters to about 30 millimeters. The dimensions from the distal end 255 to the proximal end 265 may range from about 60 millimeters or more to about 25 millimeters and may desirably be between about 40 millimeters to about 30 millimeters. These are general dimensions and it is contemplated that the dimensions may be larger or smaller.

In the practice of the disclosed technology the balloon component may be formed from thermoplastic polyurethane polymers, thermoplastic polyolefin elastomers, thermoplastic polyolefin block copolymers, SBS di-block elastomers, SEBS tri-block elastomers, polyvinyl chloride (PVC), polyethylene terephthalate (PET) and blends and mixtures thereof. More desirably, polyurethane may be used because it has been found to cause less irritation to tissues than other materials.

Useful polyurethanes include those from the Dow Chemical Company (Dow Plastics) available under the tradename Pellethane®. Pellethane® thermoplastic polyurethane elastomer is available in a number of grades and hardnesses and the particular one selected for a specific use will depend on the properties desired in the final product. The hardness of a polymer, for example, is an attribute that may be varied to meet the requirements of various applications.

FIG. 9 is an illustration of an elongated cuffed tracheostomy tube 50 as generally described in U.S. Pat. No. 6,612,305 which is composed of a tube 55 and an inflatable cuff 60 and has a flange 51 on its proximal end lying next to the skin of the throat. The proximal end of the tube is connected to a ventilator hose 52. In this configuration the cuff 60 expands not only around the tube 55, as do the conventionally available tracheostomy devices, but also cranially to it and to the stoma. One can clearly visualize that the planes P and R for this device intersect at a point on the surface of the tube that is on or within the balloon, meaning that this device blocks completely the access of secretions to the stoma.

The device further may include means for inflating and deflating the balloon (not shown). These means for inflating and deflating the balloon may be conventional flexible conduits and fittings. An yet another aspect, the tracheostomy tube device may further include a flexible conduit along the tube and the balloon such that secretions eventually accumulating cranially to the tracheal stoma can be removed.

Returning again to FIGS. 1 and 2, one advantage of having an inflatable balloon cuff having walls that are 30 micrometers or less (e.g., from 15 to 30 micrometers in the upper region to about 5 to 15 micrometers in the lower region) or even much less is illustrated in the drawings. In particular, FIG. 1 illustrates a deflated conventional balloon cuff 3 having a conventional wall thickness that is much greater than 30 micrometers. It is evident that the balloon cuff provides substantial additional material that needs to pass through the tracheal stoma during insertion when compared to the disclosed device. In contrast, FIG. 2 illustrates a deflated thin balloon cuff 4 according to an aspect of the present disclosure. This thin balloon cuff has a wall thickness of 30 micron or less and is able to hold tightly to the shaft of the tube presenting little additional material that needs to pass through the tracheal stoma during insertion.

In order to illustrate the above hypothesis, a tracheostomy device having a tube and balloon according to this disclosure and a commercial Shiley® tracheostomy device having a tube and balloon (available from Tyco Healthcare's Nellcor Puritan Bennett division) were tested by attempting to pass them through a simple template about 2 mm thick and having round holes of varying sizes, until the size through which they would not pass was determined. The instantly disclosed device was designated a size 8 by the manufacturer, having an inner tube diameter (ID) of 8 mm, an outer tube diameter of 11.3 mm and a balloon thickness of 30 microns or less and the balloon was formed from a Dow polyurethane designated Pellethane® 2363-90A which has a durometer hardness of 90A (ASTM D-2240). This polyurethane has a softening temperature of 110° C. (ASTM D-790) and a melt index of 30 g/10 min. at 224° C., 2160 g (ASTM D-1238). The Shiley® device was designated a size 6 by the manufacturer, having an ID of 6.4 mm, an OD of 10.8 mm and a balloon thickness much greater than 30 microns. It is believed the Shiley® device balloon had a thickness of at least 60 microns.

The largest size hole on the test template was 12.7 mm, a size through which the Shiley® device tube and balloon would not pass because the un-inflated balloon bunched up above the hole and made passage impossible. The application of great force may have made the Shiley® device balloon pass through the 12.7 mm hole but it was judged that the balloon would have been damaged had that amount of force been applied. The disclosed tube and balloon passed easily through the 12.7 mm hole, through the 12.3 mm hole and through the 11.91 mm hole (½, 31/64 and 15/32 inches respectively). The inventive tube and balloon would not pass easily through the next smaller hole; 29/64 inches or 11.51 mm.

The results show that the disclosed device passed through a much smaller hole than the commercial device, even though the disclosed device tube had an outer diameter 0.5 mm larger than the commercial device tube. The ability to insert a tracheostomy device into a smaller hole with ease clearly is an advantage in performing a tracheostomy. This represents a potential ability to perform a tracheostomy on smaller (e.g. younger) patients and on all patients with less trauma. The inventive balloon lies tight to the shaft, as illustrated in FIG. 2, because of its thinness, and provides an ease of insertion superior to comparable commercially available devices such as the one tested, that use a balloon greater than 30 microns in thickness. The term “comparable” as used herein means a tracheostomy device that has a tube with about the same outer diameter as the reference tube, but has a different balloon thickness.

The present invention also encompasses a system for reducing the force to insert a balloon cuffed distal end portion of a tracheostomy tube device through a patient's tracheal stoma and into the tracheal lumen such that the distal end portion of the tube extends in a first direction within the tracheal lumen when the proximal end portion extends in a second direction through the tracheal stoma, the distal end portion of the tube being enveloped by an inflatable balloon that is adapted to seal the trachea upon inflation of the balloon.

The system employs a tracheostomy tube device that includes a hollow tube having a proximal end portion, a distal end portion, and a bend region intermediate of the end portions. The distal end portion of the tube is arranged for insertion through a patient's tracheal stoma and into the tracheal lumen such that the distal end portion of the tube extends in a first direction within the tracheal lumen when the proximal end portion extends in a second direction through the tracheal stoma. The proximal end portion of the tube defines a proximal plane of the device;

Another feature of the system is an inflatable balloon that envelops the tube. The balloon has a distal balloon portion substantially centered about and attached to the distal end portion of the tube. The balloon also has a proximal balloon portion attached to the bend region of the tube and positioned substantially off-center about the bend region below the proximal plane of the tube. Upon inflation, this configuration provides for expansion of the balloon around the distal end portion of the tube and the proximal end portion of the device below the proximal plane of the device to seal the trachea below the tracheal stoma without sealing the tracheal stoma.

An important feature of the system is that the balloon has walls with a thickness of 30 microns or less. This is thought to provide for insertion into the tracheal stoma with greater ease than a comparable balloon having walls with a thickness of greater than 30 microns. The system further includes means for inflating and deflating the balloon.

The balloon can have features as generally described above including, but not limited to, having walls in different portions of the balloon of a non-uniform thickness.

This application is one of a group of commonly assigned patent application which are being filed on the same day. The group includes application Ser. No. ______ (attorney docket no. 64391725U.S.02) in the name of Brian Cuevas and is entitled “Improved Balloon Cuff Tracheostomy Tube”; application Ser. No. ______ (attorney docket no. 64391725US03) in the name of Brian Cuevas and is entitled “Improved Balloon Cuff Tracheostomy Tube with Greater Ease of Insertion”; application Ser. No. ______ (attorney docket no. 64391725U.S.04) in the name of Brian Cuevas and is entitled “A Tubular Workpiece for Producing an Improved Balloon Cuff Tracheostomy Tube”; application Ser. No. ______ (attorney docket no. 64391725U.S.05) in the name of Brian Cuevas and is entitled “A Method of Making an Improved Balloon Cuff Tracheostomy Tube”;

Modifications and variations of the present invention will be obvious to those of skill in the art from the foregoing detailed description. Such modifications and variations are intended to come within the scope of the following claims.

I claim:

1. A tracheostomy tube device comprising:
a hollow tube having a proximal end portion, a distal end portion, and a bend region intermediate of the end portions, wherein the distal end portion is arranged for insertion through a patient's throat and tracheal stoma and into the tracheal lumen such that the distal end portion of the tube extends in a first direction within the tracheal lumen when the proximal end portion extends in a second direction through the tracheal stoma, the proximal end portion defining a proximal plane of the device;
an inflatable balloon enveloping the tube, the balloon having a distal balloon portion substantially centered about and attached to the distal end portion of the tube and a proximal balloon portion attached to the bend region of the tube and positioned substantially off-center about the bend region below the proximal plane of the device, which upon inflation provides for expansion of the balloon around the distal end portion of the tube and the proximal end portion of the tube below the proximal plane of the device to seal the trachea below the tracheal stoma without sealing the tracheal stoma;
said balloon having walls with a thickness of 30 microns or less, thus providing for insertion into the tracheal stoma with greater ease than a comparable balloon having walls with a thickness of greater than 30 microns; and
means for inflating and deflating the balloon.

2. The tracheostomy tube device of claim 1, where in the balloon has walls having a non-uniform thickness.

3. The tracheostomy tube device of claim 2, wherein a first portion of the balloon has walls having a thickness of about 20 to 30 micrometers and a second portion of the balloon has walls having a thickness of about 5 to about 15 micrometers.

4. The tracheostomy tube device of claim 3, wherein the first portion of the balloon is the portion of the balloon contacting the upper portion of a cross-sectional region of the tracheal lumen and the second portion of the balloon is the portion of the balloon contacting the lower portion of the same cross-sectional region of the tracheal lumen.

5. The tracheostomy tube device of claim 1, wherein the means for inflating and deflating the balloon comprises a flexible conduit in communication with the balloon.

6. The tracheostomy tube device of claim 1, further comprising a flexible conduit along both the tube and the balloon connects the posterior, or cranial, aspect of the balloon, within the first direction inside the trachea, to the environment, such that secretions eventually accumulating cranially to the tracheal stoma can be removed.

7. The tracheostomy tube device of claim 1, wherein the balloon comprises thermoplastic polyurethane polymers, thermoplastic polyolefin elastomers, thermoplastic polyolefin block copolymers, SBS di-block elastomers, SEBS tri-block elastomers and blends and mixtures thereof.

8. A tracheostomy device for insertion into a trachea through a tracheal stoma, comprising a tube and balloon, wherein said balloon upon inflation seals the trachea below the tracheal stoma without sealing the tracheal stoma, said balloon is 30 microns or less in thickness and is positioned substantially off-center about a bend region of said tube, and wherein said balloon allows insertion into a smaller stoma than is needed for a comparable device having a balloon thickness of greater than 30 microns.

9. The device of claim 8 wherein the balloon has walls having a non-uniform thickness.

10. The device of claim 10 wherein a first portion of the balloon has walls having a thickness of about 20 to 30 micrometers and a second portion of the balloon has walls having a thickness of about 5 to about 15 micrometers.

11. The device of claim 8 further comprising a suction lumen along said tube and balloon such that secretions accumulating cranially to the tracheal stoma can be removed.

12. A system for reducing the force to insert a balloon cuffed distal end portion of a tracheostomy tube device through a patient's tracheal stoma and into the tracheal lumen such that the distal end portion of the tube extends in a first direction within the tracheal lumen when the proximal end portion extends in a second direction through the tracheal stoma, the distal end portion of the tube being enveloped by an inflatable balloon that is adapted to seal the trachea upon inflation of the balloon, the tracheostomy tube device comprising:
a hollow tube having a proximal end portion, a distal end portion, and a bend region intermediate of the end portions, wherein the distal end portion is arranged for insertion through a patient's throat and tracheal stoma and into the tracheal lumen such that the distal end portion of the tube extends in a first direction within the tracheal lumen when the proximal end portion extends in a second direction through the tracheal stoma, the proximal end portion defining a proximal plane of the device;
an inflatable balloon enveloping the tube, the balloon having a distal balloon portion substantially centered about and attached to the distal end portion of the tube and a proximal balloon portion attached to the bend region of the tube and positioned substantially off-center about the bend region below the proximal plane of the device, which upon inflation provides for expansion of the balloon around the distal end portion of the tube and the proximal end portion of the tube below the proximal plane of the device to seal the trachea below the tracheal stoma without sealing the tracheal stoma;
the balloon further having walls with a thickness of 30 microns or less, thus providing for insertion into the tracheal stoma with greater ease than a comparable balloon having walls with a thickness of greater than 30 microns; and
means for inflating and deflating the balloon.

13. The tracheostomy tube device of claim 12, where in the balloon has walls having a non-uniform thickness.

14. The tracheostomy tube device of claim 13, wherein a first portion of the balloon has walls having a thickness of about 20 to 30 micrometers and a second portion of the balloon has walls having a thickness of about 5 to about 15 micrometers.

15. The tracheostomy tube device of claim 14, wherein the first portion of the balloon is the portion of the balloon contacting the upper portion of a cross-sectional region of the tracheal lumen and the second portion of the balloon is the portion of the balloon contacting the lower portion of the same cross-sectional region of the tracheal lumen.
